(19) United States
(12) Patent Application Publication
(10) Pub. No.: US 2023/0130789 A1
(43) Pub. Date: Apr. 27, 2023

(54) METHOD FOR PROVIDING CONTROL DATA OF A LASER DEVICE FOR THE NON-DESTRUCTIVE LASER-INDUCED PROPERTY CHANGE OF A POLYMER STRUCTURE

(71) Applicant: SCHWIND EYE-TECH-SOLUTIONS GMBH, Kleinostheim (DE)

(72) Inventors: Pascal NAUBEREIT, Aschaffenburg (DE); Samuel ARBA MOSQUERA, Aschaffenburg (DE)

(21) Appl. No.: 17/951,322

(22) Filed: Sep. 23, 2022

(30) Foreign Application Priority Data
Oct. 21, 2021 (DE) 10 2021 127 399.4

(51) Int. Cl. H01S 3/10; C08J 7/12; C08J 3/28; C08J 3/24; A61F 9/008; H01S 3/11; B29C 71/04

(52) U.S. Cl. CPC H01S 3/10038; C08J 7/123; C08J 3/28; C08J 3/24; A61F 9/008; H01S 3/11; H01S 3/10069; B29C 71/04; A61F 2009/00842; A61F 2009/00872; G02B 1/041

(57) ABSTRACT

The invention relates to a method for providing control data of a laser device (10) for the non-destructive laser-induced property change of a polymer structure (14). As steps, the method includes ascertaining (S10) a respective irradiation parameter range for preset irradiation parameters of the laser device (10) by means of an irradiation model, wherein a property change model is provided in the irradiation model, in which a caused property change of the polymer structure (14) is modelled depending on the irradiation parameters, wherein a destruction threshold value model is provided in the irradiation model, in which at least one threshold value for a laser-induced optical breakthrough of the polymer structure is modelled depending on the irradiation parameters, and wherein the caused property change from the property change model is optimized while limiting by the threshold value from the destruction threshold value model for ascertaining the irradiation parameter ranges.

### FIELD

The invention relates to a method for providing control data of a laser device for the non-destructive laser-induced property change of a polymer structure, to a laser device with a control device, which is configured to perform the method, as well as to a computer program and to a computer-readable medium.

### BACKGROUND

Methods for a non-surgical and non-destructive property change, respectively, of a polymer structure are known in the ophthalmology. Hereto, for example the laser-induced refractive index change (LIRIC) is classed among it, in which the polymer structure of an artificial or biological tissue is changed by means of laser irradiation such that a phase change of the passing light and thus a refractive index change for correction of visual disorders can be achieved. By non-destructive, it is meant that the macroscopic shape of the polymer structure, for example of a cornea or an artificial lens, is not changed. This means, a lenticule is not cut out of the polymer structure to achieve the refractive index change. Therefore, this method can also be referred to as non-surgical. A further exemplary method for a non-destructive laser-induced property change is the so-called “cross-linking” method, in which a cross-linking of the polymer structure is generated by means of laser irradiation, which increases a mechanical stability.

### SUMMARY

The invention is based on the object to provide irradiation parameter ranges for a non-destructive laser-induced property change of a polymer structure.

This object is solved by the method according to the invention, the devices according to the invention and the computer program according to the invention. Advantageous developments are specified by the dependent claims, the description and the figures.

The invention is based on the idea that an irradiation model is created for the determination of the irradiation parameter ranges, in which the property change is modelled depending on the irradiation parameters, wherein the irradiation parameters are limited by a respective threshold value, which is obtained from a further model, for example a thermal model and/or a model modelling an optical breakthrough.

A first aspect of the invention relates to a method for providing control data of a laser device for the non-destructive laser-induced property change of a polymer structure. For example, the method can be performed by a control device or by a computing device. By a control device, an appliance, an appliance component or an appliance group can be understood, which is configured and arranged for receiving and evaluating signals as well as for generating control signals. For example, the control device can be configured as a control appliance or control chip or computer program. As the steps, the method includes ascertaining a respective irradiation parameter range for preset irradiation parameters of the laser device by means of an irradiation model, wherein a property change model is provided in the irradiation model, in which a caused property change of the polymer structure is modelled depending on the irradiation parameters, wherein a destruction threshold model is provided in the irradiation model, in which at least one threshold value for a laser-induced damage of the polymer structure is modelled depending on the irradiation parameters, and wherein the caused property change from the property change model is calculated or optimized while limiting by the threshold value from the destruction threshold value model for ascertaining the irradiation parameter ranges. Finally, the control data can be provided for the laser device, wherein the control data includes the ascertained irradiation parameter ranges.

In other words, the irradiation parameter ranges can be determined by means of an irradiation model to achieve a non-destructive laser-induced property change of a polymer structure. In this context, non-destructive means that a macroscopic shape of the polymer structure remains stable, wherein the polymer structure can be biological or artificial. For example, an artificial optical lens and/or a cornea/lens of a human or animal eye can be built of a polymer structure.

Irradiation parameters, which are adjusted for the property change and for which the respective irradiation parameter ranges are ascertained, can for example include a numerical aperture, a pulse length, an energy, a wavelength, a repetition frequency, a pulse path distance and/or a pulse distance between individual pulses. In the irradiation model, a property change model can be provided, in which it is mathematically/physically modelled, which property change the polymer structure experiences with preset irradiation parameters. For example, the phase change in the polymer structure can be modelled depending on the irradiation parameters in a laser-induced refractive index change (LIRIC). In addition thereto, a destruction threshold model can be provided, by means of which a laser-induced damage of the polymer structure, in particular an optical breakthrough or a cavitation bubble and/or a thermal denaturation, is modelled depending on the respective irradiation parameters. That means, two antagonistic models are provided, wherein one describes the effect of the property change and the other one describes a destruction threshold of the polymer structure. The preferred irradiation parameter ranges can then be determined from an optimization of these two models, wherein the irradiation parameters are preferably optimized such that the property change is maximized without generating a damage of the polymer structure. The irradiation parameter ranges thus obtained can then be provided for the control of the laser device by means of the control data.

By the invention, the advantage arises that a “corridor” of all of the irradiation parameters can be determined and defined such that a clear property change effect can be achieved without damaging the polymer structure. Thus, an improved treatment of biological polymer structures can be achieved and cost can be reduced in the processing of artificial polymer structures.

The invention also includes forms of configuration, by which additional advantages arise.

In a form of configuration, it is provided that the control data is provided for a laser-induced refractive index change (LIRIC) of the polymer structure and/or a cross-linking method of the polymer structure. In the laser-induced refractive index change (LIRIC), a refractive index of a human or animal eye is changed by laser irradiation without removing tissue from the eye. Herein, a lens is incorporated in a cornea in that a molecular structure of the polymer structure is changed. Furthermore, the laser-induced refractive index change can also be performed on artificial structures to generate lens properties for them. The cross-linking method is an application, in which not only a visual disorder can be treated, but also diverse other disease patterns can be treated, for example a keratoconus. In the cross-linking method, cross-linking connections of the polymer structure are increased, which increases a stability. In this form of configuration, the advantage arises that the method can be provided for preferred forms of application.

A further form of configuration provides that the control data is provided for a solid-state laser, in particular a fiber laser or crystal laser. In a solid-state laser, a crystal or a glass is doped with ions, wherein these ions provide the active medium of the solid-state laser. By optical excitations of these ions, laser radiation can then be generated, for example by diode-pumped solid-state lasers. An example for a crystal laser is a so-called yttrium-aluminum-garnet laser (YAG laser), wherein these lasers can be very expensive. Therefore, fiber lasers are preferred. By a fiber laser, an appliance, an appliance group or an appliance component is understood, which can comprise a fiber oscillator and/or a fiber amplifier. A fiber laser combines many advantages of individual laser types without having the corresponding disadvantages, wherefore the use of a fiber laser for incision-free property change methods involves considerable advantages. A fiber laser offers a required flexibility with respect to the irradiation parameters, in particular to generate variable repetition frequencies and variable/short pulse durations, it has a required stability of the irradiation parameters, in particular of an energy, pulse duration, repetition frequency and pulse shape, and has an increased freedom from maintenance. In particular, many irradiation parameters can be easier achieved with a fiber laser. By this form of configuration, the advantage arises that the ascertained irradiation parameters can be more accurately adjusted.

A further advantageous form of configuration provides that an irradiation parameter range of a numerical aperture between 0.15 and 0.35, in particular between 0.2 and 0.3, of a pulse length between 10 femtoseconds and 90 femtoseconds, in particular between 30 femtoseconds and 75 femtoseconds, of an energy between 5 nanojoules and 95 nanojoules, in particular between 20 nanojoules and 80 nanojoules, of a wavelength between 300 nanometers and 1,500 nanometers, in particular between 900 nanometers and 1,100 nanometers, and of a repetition frequency between 100 kilohertz and 100 megahertz, in particular between 5 megahertz and 75 megahertz, is provided as the control data for a laser-induced refractive index change (LIRIC). In other words, for the previously mentioned irradiation parameters, the indicated irradiation parameter ranges are provided for an optimized laser-induced refractive index change, which can subsequently be used for a control of the laser device, wherein the respective limit values are also included in the respective ranges. Hereby, the intermediate values within the respective irradiation parameter ranges are also to be considered as disclosed.

A further form of configuration provides that a pulse distance along a scanning direction between 1 nanometer and 10 micrometers, in particular between 10 nanometers and 1 micrometer, is provided for the control data. This means that the irradiation parameter range for the pulse distance is between the above indicated limits, wherein the limit values are also included in the respective range. The irradiation parameter ranges mentioned here can preferably also be used with the previously mentioned irradiation parameter ranges to achieve a preferred refractive index change. The pulse distance can for example be adjusted by means of a suitable adjustment of the repetition frequency and a scanning speed.

Preferably, it is provided that a pulse path distance of respectively adjacent laser pulse paths between 10 nanometers and 50 micrometers, in particular between 50 nanometers and 5 micrometers, is provided for the control data. This means that the pulse path distance is adjusted by means of the previously mentioned irradiation parameter ranges in spatial directions, which do not extend along the scanning direction, to obtain optimum irradiation parameters, in particular for the refractive index change of the polymer structure.

A further form of configuration provides that in ascertaining the irradiation parameter ranges, the irradiation parameters are delimited from the threshold value by a preset factor. In other words, in the optimization of the models, in particular in the destruction threshold value model, it can also be provided that the irradiation parameters are delimited from the threshold value by a preset factor, wherein the factor can be greater than or equal to 1 in a division of the threshold value by the factor and can be less than 1 in a multiplication of the threshold value by the factor. This means that a safety distance from the threshold value can be taken into account by the preset factor such that the irradiation parameters remain below the threshold value. By this form of configuration, a safety in the irradiation of the polymer structure can be increased.

A further form of configuration provides that the control data is provided for a property change of a biopolymer, in particular of a cornea of a human or animal eye. In other words, the polymer structure is a biopolymer, for which the irradiation parameter ranges are ascertained. Hereby, the advantage arises that a treatment of a human or animal eye can be improved.

In a further form of configuration, it is provided that the control data is provided for a property change of a plastic polymer, in particular for generating artificial lenses. In other words, the polymer structure is a plastic polymer. By this form of configuration, artificial lenses can in particular be generated in improved manner.

A further preferred embodiment provides that an energy and/or a laser pulse distance for the generation of the property change of the polymer structure are provided by a variably changeable value within the respective irradiation parameter range in the control data, wherein the further irradiation parameters are kept constant within their irradiation parameters. This means that for example the energy and/or the laser pulse distance can be variable for different property changes, in particular for a refractive index change depending on an irradiation position, within the respective ascertained irradiation parameter ranges and the further irradiation parameters, for example the numerical aperture, pulse length and/or wavelength, are therein fixed to a set value within the respective irradiation parameter ranges. Thus, the desired effect can be simpler and better achieved.

A second aspect of the invention relates to a method for controlling a laser device for a non-destructive laser-induced refractive index change (LIRIC) of a polymer structure, comprising controlling the laser device by means of a control device such that it emits pulsed laser pulses in a shot sequence in a preset pattern into the polymer structure, wherein the laser pulses for the non-destructive refractive index change of the polymer structure are emitted with a numerical aperture between 0.15 and 0.35, a pulse length between 10 fs and 90 fs, an energy between 5 nJ and 95 nJ, a wavelength between 300 nm and 1500 nm, and a repetition frequency between 100 kHz and 100 MHz, wherein the wavelength can be particularly preferably selected between 800 nm and 1450 nm, in particular between 900 nm and 1100 nm, to operate the laser in a preferred wavelength range for the laser-induced refractive index change. In other words, a method is provided, in which the laser device is controlled by means of the control data, which has been provided in the preceding aspect. Particularly preferably, the wavelength can be between 800 nm and 1450 nm. Further advantageous configurations of this inventive aspect can be taken from the configurations of the first aspect of the invention, wherein the same advantages and possibilities of variation arise.

A third aspect of the present invention relates to a control device, which is configured to perform one of the above described embodiments of the method according to the invention. The above cited advantages arise. The control device can for example be configured as a control chip, control appliance or application program (“app”). The control device can preferably comprise a processor device and/or a data storage. By a processor device, an appliance or an appliance component for electronic data processing is understood. The processor device can for example comprise at least one microcontroller and/or at least one microprocessor. Preferably, a program code for performing the method according to the invention can be stored on the optional data storage. The program code can then be configured, upon execution by the processor device, to cause the control device to perform one of the above described embodiments of the method according to the invention.

A fourth aspect of the present invention relates to a laser device with at least one control device. The control device can be configured to perform one of the above described embodiments of the method according to the invention. The above cited advantages arise. The control device can for example be configured as a control chip, control appliance or application program (“app”). The control device can preferably comprise a processor device and/or a data storage. By a processor device, an appliance or an appliance component for electronic data processing is understood. The processor device can for example comprise at least one microcontroller and/or at least one microprocessor. Preferably, a program code for performing the method according to the invention can be stored on the optional data storage. The program code can then be configured, upon execution by the processor device, to cause the control device to perform one of the above described embodiments of the method according to the invention. The above described advantages arise.

In an advantageous configuration of the laser device, it is provided that the laser device comprises a solid-state laser, in particular a fiber laser. By a fiber laser, an appliance, an appliance group or an appliance component is understood, which includes a fiber oscillator and/or a fiber amplifier. A fiber laser combines many advantages of the individual laser types without having the corresponding disadvantages, wherefore the use of a fiber laser for the non-destructive laser-induced property change of a polymer structure involves considerable advantages. A fiber laser offers the required flexibility with respect to the parameter space (in particular for example variable repetition rate and variable/short pulse duration), the required stability of the parameters (in particular for example pulse energy, pulse duration, repetition rate and pulse shape) and an increased freedom from maintenance (for example an air cooling (“air-cooled”) and a long lifetime). The flexibility with respect to the parameter space arises in that many parameters can be easier achieved with a fiber laser. Therein, a fiber oscillator and a fiber amplifier can for example be encompassed by the fiber laser according to the invention, but for example also a fiber oscillator and a solid-state amplifier. By the employment of the fiber laser, which is only used for removing lenticules in the prior art up to now, thus, for surgical and thereby invasive methods, a non-surgical or incision-free method is optimized by the invention.

In a further advantageous configuration of the laser device according to the invention, the laser device can be suitable to emit laser pulses in a wavelength range between 300 nm and 1500 nm, preferably between 900 nm and 1100 nm, at a respective pulse duration between 10 fs and 90 fs, preferably between 30 fs and 75 fs, and a repetition frequency of greater than 10 kilohertz (kHz), preferably between 100 kHz and 100 megahertz (MHz). The already above mentioned advantages arise.

In further advantageous configurations of the laser device according to the invention, the control device can comprise at least one storage device for at least temporary storage of at least one control dataset, wherein the control dataset or datasets include(s) control data for positioning and/or for focusing and/or for irradiation parameter adjustment of individual laser pulses; and can comprise at least one beam device for beam guidance and/or beam shaping and/or beam deflection and/or beam focusing of a laser beam of the laser. Therein, the mentioned control datasets are preferably generated based on the first aspect of the invention and/or a measured topography and/or pachymetry and/or morphology.

Further features and the advantages thereof can be taken from the descriptions of the first inventive aspect, wherein advantageous configurations of each inventive aspect are to be regarded as advantageous configurations of the respectively other inventive aspect.

A fifth aspect of the invention relates to a computer program including commands, which cause the laser device according to the third inventive aspect to execute the method steps according to the first and/or second inventive aspects.

A sixth aspect of the invention relates to a computer-readable medium, on which the computer program according to the fifth inventive aspect is stored. Further features and the advantages thereof can be taken from the descriptions of the first to fourth inventive aspects, wherein advantageous configurations of each inventive aspect are to be regarded as advantageous configurations of the respectively other inventive aspect.

### BRIEF DESCRIPTION OF THE DRAWINGS

Further features of the invention are apparent from the claims, the figures and the description of figures. The features and feature combinations mentioned above in the description as well as the features and feature combinations mentioned below in the description of figures and/or shown in the figures alone are usable not only in the respectively specified combination, but also in other combinations without departing from the scope of the invention. Thus, implementations are also to be considered as encompassed and disclosed by the invention, which are not explicitly shown in the figures and explained, but arise from and can be generated by separated feature combinations from the explained implementations. Implementations and feature combinations are also to be considered as disclosed, which thus do not comprise all of the features of an originally formulated independent claim. Moreover, implementations and feature combinations are to be considered as disclosed, in particular by the implementations set out above, which extend beyond or deviate from the feature combinations set out in the relations of the claims.

FIG. 1 depicts a schematic representation of a laser device according to an exemplary embodiment.

FIG. 2 depicts a schematic method diagram according to an exemplary embodiment.

In the figures, identical or functionally identical elements are provided with the same reference characters.

### DETAILED DESCRIPTION

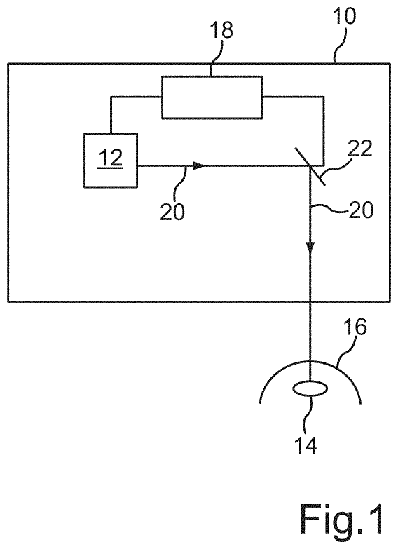

FIG. 1 shows a schematic representation of a laser device 10 with a laser 12, in particular a solid-state laser, for the non-destructive laser-induced property change of a polymer structure 14. In this embodiment, the polymer structure 14 can be a biopolymer, in particular an area of a cornea 14 of a human or animal eye 16, and the property change can be a laser-induced refractive index change (LIRIC) of the cornea 14. A laser pulse sequence, a laser pulse distribution and irradiation parameters for refractive index change of the cornea 14 can be provided in the form of control data by a control device 18, such that the laser 12 emits pulsed laser pulses in laser pulse positions preset by the control data with irradiation parameters provided by the control data to achieve the refractive index change. Alternatively, the control device 18 can be a control device 18 external with respect to the laser device 10.

Furthermore, FIG. 1 shows that the laser beam 20 generated by the laser 12 can be deflected towards the eye 16 by means of a beam deflection device 22, namely a beam deflection apparatus such as for example a rotation scanner, to generate the refractive index change in the cornea 14. The beam deflection apparatus 22 can also be controlled by the control device 18.

Preferably, the illustrated laser 12 can be a fiber laser, which is at least formed to emit laser pulses in a wavelength range between 300 nanometers and 1500 nanometers, preferably between 900 nanometers and 1100 nanometers, at a respective pulse duration between 10 femtoseconds and 90 femtoseconds, preferably between 30 femtoseconds and 75 femtoseconds, and a repetition frequency of greater than 10 kilohertz, preferably between 100 kilohertz and 100 megahertz.

Optionally, the control device 18 additionally comprises a storage device (not illustrated) for at least temporary storage of at least one control dataset, wherein the control dataset or datasets include(s) control data for irradiation parameter adjustment, positioning and/or focusing of individual laser pulses in the eye 16.

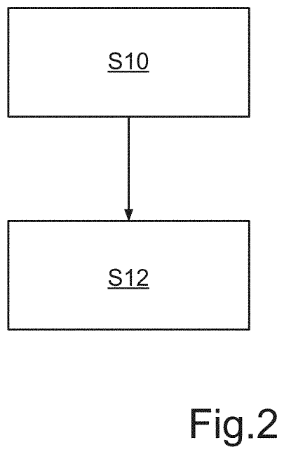

In a laser-induced refractive index change, but also in other non-surgical methods for property change of the polymer structure 14, such as for example the cross-linking method, it is provided to achieve a maximized effect of the property change per laser pulse without therein damaging the polymer structure 14. In particular with a too high energy density, cavitation bubbles can arise, which is to be avoided. In order to obtain the optimum irradiation parameters, the control device 18 can therefore perform the method shown in FIG. 2.

In FIG. 2, a schematic method diagram for providing control data for the laser device 10 for non-destructive laser-induced property change of a polymer structure is illustrated, wherein the polymer structure can be a cornea 14 of an eye 16 in this embodiment and the property change can be a laser-induced refractive index change (LIRIC).

In a step S10, an optimum irradiation parameter range for a respective irradiation parameter can be determined by the control device 18, wherein irradiation parameters can for example be a numerical aperture, a pulse length, an energy, a wavelength, a repetition frequency, a pulse distance and/or a pulse path distance. For determining the optimum irradiation parameter ranges, an irradiation model can be used, in which a property change model and a destruction threshold value model are provided. In this embodiment, the property change model can be a LIRIC model, which describes an induced phase change by laser irradiation. In particular, the phase change can be determined by means of the formula

Δϕ=γ·P_{avg}^{m}·NA^{2(m-2)}·m^{m-2}·υ^{1-m}·τ^{1-m}·λ_{write}^{3-2m}·λ_{read}⁻¹·S⁻¹·t⁻¹

wherein ΔΦ is the induced phase change, γ is a material constant, P_{avg} is the average power of the laser, NA is the numerical aperture of the laser device 10, m is the order of the multi-photon absorption, v is the repetition frequency of the laser 12, τ is the pulse duration, λ_{read} is the wavelength of the laser radiation 20, λ_{write} is the wavelength, for which the phase change is to be provided, S is a scanning speed and t is a pulse path distance.

The destruction threshold value model, which describes a threshold value for a laser-induced damage of the polymer structure, can be a modelling of an optical breakthrough in this embodiment, wherein the model of the optical breakthrough can be given by the formula

E_{Th}∝√³τ·λ·√m·M2²·NA⁻²

wherein E_{TH} represents the pulse energy threshold value for the optical breakthrough and M2 represents a beam quality. Alternatively or additionally, further destruction threshold value models can be taken into account, which describe a damage of the polymer structure, such as for example thermal models.

In order to ascertain the optimum irradiation parameter ranges from this irradiation model, it is preferably provided to maximize the phase change without initiating the optical breakthrough.

As a step S12, the control data thus ascertained can then be provided for the laser device 10, by means of which the control device 18 for example can control the laser 12 and the beam deflection device 22 for refractive index change. For the laser-induced refractive index change, an irradiation parameter range of a numerical aperture between 0.15 and 0.35, in particular between 0.2 and 0.3, of a pulse length between 10 femtoseconds and 90 femtoseconds, in particular between 30 femtoseconds and 75 femtoseconds, of an energy between 5 nanojoules and 95 nanojoules, in particular between 20 nanojoules and 80 nanojoules, of a wavelength between 300 nanometers and 1,500 nanometers, in particular between 900 nanometers and 1,100 nanometers, and of a repetition frequency between 100 kilohertz and 100 megahertz, in particular between 5 megahertz and 75 megahertz, can preferably be provided in the control data for controlling the laser device 10. Furthermore, a pulse distance along a scanning direction between 1 nanometer and 10 micrometers, in particular between 10 nanometers and 1 micrometer, and a pulse path distance of respectively adjacent laser pulse paths between 10 nanometers and 50 micrometers, in particular between 50 nanometers and 5 micrometers, can preferably be provided by means of the beam deflection device 22.

For the suitable control of the laser device 10, for example to achieve a refractive index change, it can preferably be provided that only the energy and/or a laser pulse distance are changed within the respective irradiation parameter ranges depending on an irradiation position in the cornea 14 in the control data, wherein a value is respectively selected from the further irradiation parameter ranges and kept constant. Thus, the suitable refractive index change can be obtained at each location of the cornea 14 without generating cavitation bubbles.

The previously shown embodiment is only one of multiple examples, in which a property change, in particular a laser-induced refractive index change, in a biopolymer, in particular the cornea 14, can be generated. Alternatively or additionally, a cross-linking method can also be performed as the property change, wherein the property change model can for example be adapted hereto. Furthermore, the polymer structure can also be a plastic polymer, in particular for generating artificial lenses.

Overall, the examples show, how optimized irradiation parameters for a non-surgical method can be provided by the invention.

1. A method for providing control data of a laser device for the non-destructive laser-induced property change of a polymer structure, comprising:
ascertaining a respective irradiation parameter range for preset irradiation parameters of the laser device by means of an irradiation model;
wherein a property change model is provided in the irradiation model, in which a caused property change of the polymer structure is modelled depending on the irradiation parameters;
wherein a destruction threshold value model is provided in the irradiation model, in which at least one threshold value for a laser-induced damage of the polymer structure is modelled depending on the irradiation parameters; and
wherein the caused property change from the property change model is optimized while being limited by the threshold value from the destruction threshold value model for ascertaining the irradiation parameter ranges; and
providing the control data for the laser device, which includes the ascertained irradiation parameter ranges.

2. The method according to claim 1, wherein the control data is provided for a laser-induced refractive index change (LIRIC) of the polymer structure and/or a cross-linking method of the polymer structure.

3. The method according to claim 1, wherein the control data is provided for a solid-state laser, in particular a fiber laser or crystal laser.

4. The method according to claim 1, wherein for a laser-induced refractive index change (LIRIC), an irradiation parameter range
of a numerical aperture between 0.15 and 0.35, in particular between 0.2 and 0.3;
of a pulse length between 10 fs and 90 fs, in particular between 30 fs and 75 fs;
of an energy between 5 nJ and 95 nJ, in particular between 20 nJ and 80 nJ;
of a wavelength between 300 nm and 1450 nm, in particular between 900 nm and 1100 nm; and
of a repetition frequency between 100 kHz and 100 MHz, in particular between 5 MHz and 75 MHz
is provided as the control data.

5. The method according to claim 1, wherein a pulse distance along a scanning direction between 1 nm and 10 μm, in particular between 10 nm and 1 μm, is provided for the control data.

6. The method according to claim 1, wherein a pulse path distance of respectively adjacent laser pulse paths between 10 nm and 50 μm, in particular between 50 nm and 5 μm, is provided for the control data.

7. The method according to claim 1, wherein the irradiation parameters are delimited from the threshold value by a preset factor in ascertaining the irradiation parameter ranges.

8. The method according to claim 1, wherein the control data is provided for a property change of a biopolymer, in particular of a cornea of a human or animal eye.

9. The method according to claim 1, wherein the control data is provided for a property change of a plastic polymer, in particular for generating artificial lenses.

10. The method according to claim 1, wherein an energy and/or a laser pulse distance for the generation of the property change of the polymer structure are provided by a variably changeable value within the respective irradiation parameter range in the control data, wherein the further irradiation parameters are kept constant within their irradiation parameter ranges.

11. A method for controlling a laser device for a non-destructive laser-induced refractive index change (LIRIC) of a polymer structure, comprising:
controlling the laser device by means of a control device such that it emits pulsed laser pulses in a shot sequence in a preset pattern into the polymer structure, wherein the laser pulses are emitted for non-destructive refractive index change of the polymer structure with a numerical aperture between 0.15 and 0.35, a pulse length between 10 fs and 90 fs, an energy between 5 nJ and 95 nJ, a wavelength between 300 nm and 1500 nm, and a repetition frequency between 100 kHz and 100 MHz.

12. A laser device with a control device, which is configured to perform a method according to claim 1.

13. The laser device according to claim 12, wherein the laser device a solid-state laser, in particular a fiber laser.

14. The laser device according to claim 12, wherein the laser device is suitable to emit laser pulses in a wavelength range between 300 nm and 1500 nm, preferably between 900 nm and 1100 nm, at a respective pulse duration between 10 fs and 90 fs, preferably between 30 fs and 75 fs, and a repetition frequency of greater than 10 kHz, preferably between 100 kHz and 100 MHz.

15. The laser device according to claim 12, wherein the control device:
comprises at least one storage device for at least temporary storage of at least one control dataset, wherein the control dataset or datasets include(s) control data for positioning and/or for focusing and/or for irradiation parameter adjustment of individual laser pulses; and
includes at least one beam device for beam guidance and/or beam shaping and/or beam deflection and/or beam focusing of a laser beam of the laser device.

16. A computer program including commands, which cause a laser device according to claim 12 with a control device to execute a method according to claim 1.

17. A non-transitory computer-readable medium, on which the computer program according to claim 16 is stored.
